(19) United States
(12) Patent Application Publication
(10) Pub. No.: US 2005/0265950 A1
(43) Pub. Date: Dec. 1, 2005

(54) Cross-linked cationic copolymers comprising regulators, and use therof in cosmetic preparations for hair

(75) Inventors: Lysander Chrisstoffels, Limburgerhof (DE); Maximilian Angel, Schifferstadt (DE); Peter Hossel, Schifferstadt (DE); Klemens Mathauer, Heidelberg (DE); Claudia Wood, Weinheim (DE); Dieter Faul, Niederkirchen (DE)

Correspondence Address: CONNOLLY BOVE LODGE & HUTZ LLP, SUITE 800, 1990 M STREET NW, WASHINGTON, DC, 20036-3425, US

(73) Assignee: BASF AKTIENGESELLSCHAFT, Ludwigshafen (DE)

(21) Appl. No.: 10/524,370

(22) PCT Filed: Jul. 24, 2003
(86) PCT No.: PCT/EP03/08097

§ 371 Date: Feb. 10, 2005

(30) Foreign Application Priority Data
Aug. 12, 2002 (DE) 102 37 378.7

(51) Int. Cl. A61K 7/06; A61K 7/11

(52) U.S. Cl. 424/70.17

(57) ABSTRACT

The invention relates to the use of polymers in cosmetic preparations for hair. Said polymers can be obtained by (i) radically initiated copolymerisation of monomer mixtures consisting of (a) at least one cationic monomer or quaternisable monomer (b), optionally a water-soluble monomer, (c) optionally another radically copolymerisable monomer, (d) at least one monomer acting as a cross-linking agent and having at least two ethylenically unsaturated, non-conjugated double bonds, and (e) at least one regulator; and by (ii) subsequent quaternisation or protonation of the polymers, provided that a non-quaternised or only partially quaternised monomer is used as monomer (a).

The present invention relates to the use of polymers obtainable by free-radically initiated copolymerization of monomer mixtures of
(a) at least one cationic monomer or quaternizable monomer
(b) optionally a water-soluble monomer,
(c) optionally a further free-radically copolymerizable monomer
(d) at least one crosslinking monomer having at least two ethylenically unsaturated, nonconjugated double bonds, and
(e) at least one regulator
   and subsequent quaternization or protonation of the polymer if the monomer (a) used is a nonquaternized polymer or an only partially quaternized monomer, as active ingredients in cosmetic preparations, in hair cosmetic preparations, and to the use of these polymers as conditioning agents in cosmetic preparations.

Cationic polymers are used as conditioning agents in cosmetic formulations. Requirements for hair conditioning agents are, for example, a considerable reduction in the required combing force in wet and also dry hair, good detangling on the first combing and good compatibility with other formulation components. In addition, cationic polymers prevent electrostatic charging of the hair.

In shampoos, cationic cellulose derivatives (polyquaternium-10) or guar gum derivatives are primarily used. However, a build-up effect is observed with these compounds, i.e. the hair becomes coated with the conditioner with repeated application and feels weighted down.

For the conditioning and setting of keratin substances, such as hair, nails and skin, synthetic polymers have also been used for years. In addition, synthetic polymers are used in cosmetic formulations which comprise pigments or cosmetically effective active components as compatibility promoters for achieving a homogeneous, stable formulation.

For example, copolymers of acrylamide and dimethyldiallylammonium chloride (polyquaternium 7) are used. However, these have the disadvantage of high residual monomer contents since acrylamide and dimethyldiallylammonium chloride have unfavorable copolymerization parameters.

Despite extensive efforts, there is still a need for improvement in the case of polymers for generating elastic hairstyles with simultaneously strong hold even at high atmospheric humidity, good ability to be washed out and good feel of the hair. The need for improvement likewise consists in the case of polymers for generating readily combable, detanglable hair and for the conditioning of skin and hair in their sensorially perceptible properties, such as feel, volume, handlability etc. Also desirable are clear aqueous preparations of these polymers which are characterized, accordingly, by good compatibility with other formulation constituents.

In addition, there is a need for polymers which are suitable as conditioning agents for cosmetic preparations and which can be prepared with a high solids content. Of particular interest are polymers which have a high solids content, have a low viscosity with simultaneously good performance properties (such as, for example, combability).

It is an object of the present invention to find a cationic conditioning agent for cosmetic preparations, in particular shampoos, which does not have said disadvantages.

Quaternized polymers and their use as conditioning agents in hair care formulations are known.

Cationic polymers are often used as conditioning agents in hair cosmetic formulations. They bring about primarily an improvement in the wet combability of hair. In addition, cationic polymers prevent electrostatic charging of hair.

Thus, for example, EP-A-0 246 580 describes the use of noncrosslinked homo- and copolymers of 3-methyl-1-vinylimidazolium chlorides in cosmetic compositions. EP-A-0 544 158 and U.S. Pat. No. 4,859,756 claim the use of noncrosslinked homo- and copolymers of chloride-free, quaternized N-vinylimidazoles in cosmetic preparations. EP-A-0 715 843 discloses the use of noncrosslinked copolymers of a quaternized N-vinylimidazole, N-vinylcaprolactam and N-vinylpyrrolidone, and optionally a further comonomer in cosmetic preparations.

DE-A-28 21 239 (U.S. Pat. No. 4,348,380) and DE-A-31 06 974 describe copolymers of quaternized diallylammonium compounds in hair cosmetic preparations.

U.S. Pat. No. 5,275,809, EP-A-0 522 755, EP-A-0 521 665 and EP-A-0 521 666 disclose copolymers with dimethyldiallylammonium chloride for use in shampoos. None of the abovementioned specifications describes a crosslinked polymer.

In addition, crosslinked cationic copolymers and their use as water-soluble and water-insoluble additives in very diverse fields are also described. U.S. Pat. No. 4,806,345 describes crosslinked cationic thickeners for cosmetic formulations of quaternized dimethylaminoethyl methacrylate and acrylamide.

WO 93/25595 describes crosslinked cationic copolymers based on quaternized dialkylaminoalkyl acrylates or dialkylaminoalkylacrylamides. As an application, the use of these crosslinked copolymers as thickeners in cosmetic preparations is proposed. These polymers do not comprise regulators.

DE 3 209 224 describes the preparation of crosslinked polymers based on N-vinylpyrrolidone and (quaternized) N-vinylimidazole. These polymers are claimed for use as adsorbents and ion exchangers. They are highly crosslinked, water-insoluble, sparingly swellable and therefore not suitable as conditioning agents in cosmetic formulations.

Crosslinked agglomerated vinylimidazole copolymers are mentioned in WO 96/26229 as dye transfer inhibitors. They are highly crosslinked, water-insoluble, not very swellable and are therefore not suitable as conditioning agents in cosmetic formulations.

U.S. Pat. No. 4,058,491 discloses crosslinked cationic hydrogels of N-vinylimidazole or N-vinylpyrrolidone and a quaternized basic acrylate and other comonomers. These gels are proposed for the complexing and controlled release of anionic active substances.

DE-A-42 13 971 describes copolymers of an unsaturated carboxylic acid, quaternized vinylimidazole and optionally further monomers and a crosslinker. The polymers are proposed as thickeners and dispersants. The method of thickening by protonating a water-soluble, crosslinked aminoalkyl (meth)acrylate is described in EP-A-0 624 617 and EP-A-0 027 850.

WO 96/37525 describes the preparation of crosslinked copolymers from, inter alia, N-vinylpyrrolidone and quaternized vinylimidazoles in the presence of polymerization regulators and their use, in particular, in detergents.

WO 97/35544 describes the use of crosslinked cationic polymers with dialkylaminoalkyl (meth)acrylates or dialkylaminoalkyl(meth)acrylamides in shampoo compositions.

DE-A-197 31 907 describes the use of crosslinked cationic copolymers which contain N-vinylimidazoles in hair cosmetic formulations.

EP 0 893 117 and EP 1 064 924 describe high molecular weight crosslinked polymers which have a good conditioning effect in shampoos, while the corresponding low molecular weight noncrosslinked polymers exhibit only low effectiveness as conditioning agents.

A disadvantage of these abovementioned inventions is that the preparation of these polymers as solutions takes place with a very low solids content because otherwise the viscosities of these solutions are too high. An additional disadvantage is the production of a relatively large fraction of undissolved gel particles. This leads to a large number of performance disadvantages, such as, for example, prolonged polymerization times, and long filtration and bottling times. The low solids content results in high costs during the preparation (vessel capacities) and high transportation costs.

It is an object of the present invention to provide polymers which are suitable for cosmetic preparations, in particular hair cosmetic preparations (conditioning properties), have a satisfactory viscosity and can also be prepared with a high solids content. Of particular interest are polymers which comprise fewer gel particles than the polymers of the prior art, in particular of EP 0 893 117.

We have found that this object is achieved by the polymers according to the invention.

The use according to the invention relates to polymers obtainable by
(i) free-radically initiated copolymerization of monomer mixtures of
   (a) at least one cationic monomer or quaternizable monomer
   (b) optionally a water-soluble monomer,
   (c) optionally a further free-radically copolymerizable monomer
   (d) at least one crosslinking monomer having at least two ethylenically unsaturated, nonconjugated double bonds, and
   (e) at least one regulator
(ii) subsequent quaternization or protonation of the polymer if the monomer (a) used is a nonquaternized monomer or is an only partially quaternized monomer.

The use according to the invention relates to polymers obtainable by (i) free-radically initiated copolymerization of monomer mixtures of
(a) 1 to 99.98% by weight, preferably 2 to 70% by weight, particularly preferably 2 to 50% by weight, of at least one cationic monomer or quaternizable monomer
(b) 0 to 98.98% by weight, preferably 22 to 97.98% by weight, particularly preferably 45 to 85% by weight, of at least one water-soluble monomer,
(c) 0 to 50% by weight, preferably 0 to 40% by weight, particularly preferably 0 to 30% by weight, of a further free-radically copolymerizable monomer,
(d) 0.01 to 10% by weight, preferably 0.02 to 8% by weight, particularly preferably 0.05 to 5% by weight, of at least one crosslinking monomer having at least two ethylenically unsaturated, nonconjugated double bonds, and
(e) 0.01 to 10% by weight, preferably 0.02 to 8% by weight, particularly preferably 0.05 to 5% by weight, of a regulator
   and (ii) subsequent quaternization or protonation of the polymer if the monomer (a) used is a nonquaternized monomer or an only partially quaternized monomer, as conditioning agent for compositions in hair cosmetics.

The polymers obtainable in this way are suitable for use in hair cosmetic preparations.

The polymers obtainable in this way are suitable as conditioning agents in cosmetic preparations, in particular in skin and/or hair cosmetic preparations.

Preference is given to the use in shampoos.

Suitable monomers (a) are the N-vinylimidazole derivatives of the formula (I) in which R¹ to R³ are hydrogen, C₁-C₄-alkyl or phenyl

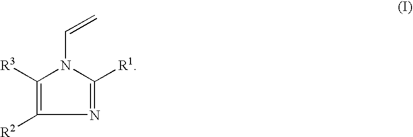

Also suitable are diallylamines of the formula (II) in which R⁴ is C₁-C₂₄-alkyl

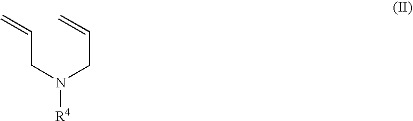

Also suitable are N,N-dialkylaminoalkylacrylates and methacrylates and N,N-dialkylaminoalkylacrylamides and -methacrylamides of the formula (III), 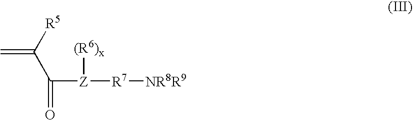
where R⁵, R⁶ independently, are a hydrogen atom or a methyl radical, R⁷ is an alkylene radical having 1 to 24 carbon atoms, optionally substituted by alkyl radicals, and R⁸, R⁹ are C₁-C₂₄ alkyl radicals. Z is a nitrogen atom together with x=1 or an oxygen atom together with x=0.

Examples of compounds of the formula (I) are given in Table 1 below:

TABLE 1

| R¹ | R² | R³ |
|---|---|---|
| H | H | H |
| Me | H | H |
| H | Me | H |
| H | H | Me |
| Me | Me | H |
| H | Me | Me |
| Me | H | Me |
| Ph | H | H |
| H | Ph | H |
| H | H | Ph |
| Ph | Me | H |
| Ph | H | Me |
| Me | Ph | H |
| H | Ph | Me |
| H | Me | Ph |
| Me | H | Ph |

Me = methyl
Ph = phenyl

Other monomers of the formula (I) which can be used are the ethyl, propyl or butyl analogs of the methyl-substituted 1-vinylimidazoles listed in Table 1.

Examples of compounds of the formula (II) are diallylamines in which R⁴ is methyl, ethyl, isopropyl or n-propyl, isobutyl, n-butyl or tert-butyl, pentyl, hexyl, heptyl, octyl, nonyl or decyl. Examples of long-chain radicals R⁴ are undecyl, dodecyl, tridecyl, pentadecyl, octadecyl and icosyl.

Examples of compounds of the formula (III) are
N,N-dimethylaminomethyl (meth)acrylate,
N,N-diethylaminomethyl (meth)acrylate,
N,N-dimethylaminoethyl (meth)acrylate,
N,N-diethylaminoethyl (meth)acrylate,
N,N-dimethylaminobutyl (meth)acrylate,
N,N-diethylaminobutyl (meth)acrylate,
N,N-dimethylaminohexyl (meth)acrylate,
N,N-dimethylaminooctyl (meth)acrylate,
N,N-dimethylaminododecyl (meth)acrylate,
N-[3-(dimethylamino)propyl]methacrylamide,
N-[3-(dimethylamino)propyl]acrylamide,
N-[3-(dimethylamino)butyl]methacrylamide,
N-[8-(dimethylamino)octyl]methacrylamide,
N-[12-(dimethylamino)dodecyl]methacrylamide,
N-[3-(diethylamino)propyl]methacrylamide,
N-[3-(diethylamino)propyl]acrylamide.

Preferred examples of monomers (a) are 3-methyl-1-vinylimidazolium chloride and methosulfate, dimethyldiallylammonium chloride and N,N-dimethylaminoethyl methacrylate and N-[3-(dimethylamino)propyl]methacrylamide which have been quaternized by methyl chloride, dimethyl sulfate or diethyl sulfate.

Particularly preferred monomers (a) are 3-methyl-1-vinylimidazolium chloride and methosulfate and dimethyldiallylammonium chloride, very particular preference being given to 3-methyl-1-vinylimidazolium chloride and methosulfate.

The monomers (a) can either be used in quaternized form as monomers or be polymerized in nonquaternized form, where, in the latter case, the resulting polymer is either quaternized or protonated.

Suitable compounds for the quaternization of the compounds of the formula (I) to (III) are, for example, alkyl halides having 1 to 24 carbon atoms in the alkyl group, e.g. methyl chloride, methyl bromide, methyl iodide, ethyl chloride, ethyl bromide, propyl chloride, hexyl chloride, dodecyl chloride, lauryl chloride and benzyl halides, in particular benzyl chloride and benzyl bromide. Further suitable quaternizing agents are dialkyl sulfates, in particular dimethyl sulfate or diethyl sulfate. The quaternization of the basic monomers of the formula (I) to (III) can also be carried out with alkylene oxides, such as ethylene oxide or propylene oxide in the presence of acids.

The quaternization of the monomer or a polymer with one of said quaternizing agents can be carried out by generally known methods.

Preferred quaternizing agents are: methyl chloride, dimethyl sulfate or diethyl sulfate.

The quaternization of the polymer can be complete or else partial. The proportion of quaternized monomers (a) within the polymer can vary over a wide range and is, for example, about 20 to 100 mol %.

Suitable compounds for the protonation are, for example, mineral acids, such as HCl, H₂SO₄, H₃PO₄, and monocarboxylic acids, such as, for example, formic acid and acetic acid, dicarboxylic acids and polyfunctional carboxylic acids, such as, for example, oxalic acid and citric acid, and all other proton-releasing compounds and substances which are able to protonate the corresponding vinylimidazole or diallylamine. In particular, water-soluble acids are suitable for the protonation.

The protonation of the polymer can either take place after the polymerization or during the formulation of the cosmetic composition, during which a physiologically compatible pH is normally established.

Protonation is understood as meaning that at least some of the protonatable groups of the polymer, preferably 20 to 100 mol %, are protonated, such that an overall cationic charge of the polymer results.

Preferred monomers of group (b) are those compounds which dissolve to more than 5% by weight in water at a temperature of 25° C. If the polymers contain monomers of group (b), then they may be present in amounts up to 98.98% by weight. Particularly preferably, they are present in amounts of from 22 to 97.98% by weight, in particular 45 to 85% by weight.

Suitable water-soluble monomers (b) different from (a) are N-vinyllactams, e.g. N-vinylpiperidone, N-vinylpyrrolidone and N-vinylcaprolactam, N-vinylacetamide, N-methyl-N-vinylacetamide, N-vinylformamide, acrylamide, methacrylamide, N,N-dimethylacrylamide, N-methylolmethacrylamide, N-vinyloxazolidone, N-vinyltriazole, hydroxyalkyl (meth)acrylates, e.g. hydroxyethyl (meth)acrylate and hydroxypropyl (meth)acrylates, or alkyl ethylene glycol (meth)acrylates having 1 to 50 ethylene glycol units in the molecule.

Particular preference is given to using N-vinyllactams as monomers (b). Very particular preference is given to N-vinylpyrrolidone.

Also suitable are N-vinylimidazoles of the formula (I) in which R¹ to R³ are hydrogen, C₁-C₄-alkyl or phenyl and which are different from monomer (a), diallylamines of the formula (II), and dialkylaminoalkyl (meth)acrylate and dialkylaminoalkyl(meth)acrylamides of the formula (III), e.g. dimethylaminoethyl methacrylate or dimethylaminopropylmethacrylamide.

Also suitable are unsaturated carboxylic acids and unsaturated anhydrides, e.g. acrylic acid, methacrylic acid, crotonic acid, itaconic acid, maleic acid, fumaric acid and their corresponding anhydrides, and unsaturated sulfonic acids, such as, for example, acrylamidomethylpropanesulfonic acid, and the salts of the unsaturated acids, such as, for example, the alkali metal or ammonium salts.

Suitable monomers (c) different from monomers (a) and (b) are C₁-C₄₀-alkyl esters of (meth)acrylic acid, where the esters are derived from linear, branched-chain or carbocyclic alcohols, e.g. methyl (meth)acrylate, ethyl (meth)acrylate, tert-butyl (meth)acrylate, isobutyl (meth)acrylate, n-butyl (meth)acrylate, stearyl (meth)acrylate, or esters of alkoxylated fatty alcohols, e.g. C₁-C₄₀-fatty alcohols, reacted with ethylene oxide, propylene oxide or butylene oxide, in particular C₁₀-C₁₈-fatty alcohols reacted with 3 to 150 ethylene oxide units. Also suitable are N-alkyl-substituted acrylamides with linear, branched-chain or carbocyclic alkyl radicals, such as N-tert-butylacrylamide, N-butylacrylamide, N-octylacrylamide, N-tert-octylacrylamide.

Also suitable are styrene, vinyl and allyl esters of C₁-C₄₀-carboxylic acids, which may be linear, branched-chain or carbocyclic, e.g. vinyl acetate, vinyl propionate, vinyl neononanoate, vinyl neoundecanoic acid, vinyl t-butylbenzoate, alkyl vinyl ethers, for example methyl vinyl ether, ethyl vinyl ether, butyl vinyl ether, stearyl vinyl ether.

Acrylamides, such as N-tert-butylacrylamide, N-butylacrylamide, N-octylacrylamide, N-tert-octylacrylamide and N-alkyl-substituted acrylamides with linear, branched-chain or carbocyclic alkyl radicals, where the alkyl radical can have the meanings given above for R⁴.

Suitable monomers (c) are, in particular, C₁ to C₂₄-, very particularly C₁ to C₁₀-, alkyl esters of (meth)acrylic acid, e.g. methyl (meth)acrylate, ethyl (meth)acrylate, tert-butyl (meth)acrylate, isobutyl (meth)acrylate, n-butyl (meth)acrylate and acrylamides, such as N-tert-butylacrylamide or N-tert-octylacrylamide.

If the polymers contain monomers of group (c), then they may be present therein in amounts up to 50% by weight, in particular up to 40% by weight, preferably up to 30% by weight.

Monomers (d), which have a crosslinking function, are compounds with at least 2 ethylenically unsaturated, nonconjugated double bonds in the molecule.

Suitable crosslinkers (d) are, for example, acrylic esters, methacrylic esters, allyl ethers or vinyl ethers of at least dihydric alcohols. The OH groups of the parent alcohols may be in completely or partially etherified or esterified form; however, the crosslinkers contain at least two ethylenically unsaturated groups.

Examples of the parent alcohols are dihydric alcohols, such as 1,2-ethanediol, 1,2-propanediol, 1,3-propanediol, 1,2-butanediol, 1,3-butanediol, 2,3-butanediol, 1,4-butanediol, but-2-ene-1,4-diol, 1,2-pentanediol, 1,5-pentanediol, 1,2-hexanediol, 1,6-hexanediol, 1,10-decanediol, 1,2-dodecanediol, 1,12-dodecanediol, neopentylglycol, 3-methylpentane-1,5-diol, 2,5-dimethyl-1,3-hexanediol, 2,2,4-trimethyl-1,3-pentanediol, 1,2-cyclohexanediol, 1,4-cyclohexanediol, 1,4-bis(hydroxymethyl)cyclohexane, neopentylglycol mono(hydroxypivalate), 2,2-bis(4-hydroxyphenyl)propane, 2,2-bis[4-(2-hydroxypropyl)phenyl]propane, diethylene glycol, triethylene glycol, tetraethylene glycol, dipropylene glycol, tripropylene glycol, tetrapropylene glycol, 3-thiopentane-1,5-diol, and polyethylene glycols, polypropylene glycols and polytetrahydrofurans with molecular weights of in each case 200 to 10 000. Apart from the hompolymers of ethylene oxide and/or propylene oxide, it is also possible to use block copolymers of ethylene oxide or propylene oxide, or copolymers which contain ethylene oxide and propylene oxide groups in incorporated form. Examples of parent alcohols with more than two OH groups are trimethylolpropane, glycerol, pentaerythritol, 1,2,5-pentanetriol, 1,2,6-hexanetriol, triethoxycyanuric acid, sorbitan, sugars, such as sucrose, glucose, mannose. The polyhydric alcohols can of course also be used following reaction with ethylene oxide or propylene oxide, as the corresponding ethoxylates or propoxylates respectively. The polyhydric alcohols can also firstly be converted into the corresponding glycidyl ethers by reaction with epichlorohydrin.

Further suitable crosslinkers are the vinyl esters or the esters of monohydric, unsaturated alcohols with ethylenically unsaturated C₃- to C₆-carboxylic acids, for example acrylic acid, methacrylic acid, itaconic acid, maleic acid or fumaric acid. Examples of such alcohols are allyl alcohol, 1-buten-3-ol, 5-hexen-1-ol, 1-octen-3-ol, 9-decen-1-ol, dicyclopentenyl alcohol, 10-undecen-1-ol, cinnamyl alcohol, citronellol, crotyl alcohol or cis-9-octadecen-1-ol. It is, however, also possible to esterify the monohydric, unsaturated alcohols with polybasic carboxylic acids, for example malonic acid, tartaric acid, trimellitic acid, phthalic acid, terephthalic acid, citric acid or succinic acid.

Further suitable crosslinkers are esters of unsaturated carboxylic acids with the above-described polyhydric alcohols, for example oleic acid, crotonic acid, cinnamic acid or 10-undecenoic acid.

Also suitable as monomers (d) are straight-chain or branched, linear or cyclic, aliphatic or aromatic hydrocarbons which have at least two double bonds which, in the case of aliphatic hydrocarbons, must not be conjugated, e.g. divinylbenzene, divinyltoluene, 1,7-octadiene, 1,9-decadiene, 4-vinyl-1-cyclohexene, trivinylcyclohexane or polybutadienes with molecular weights of from 200 to 20 000.

Also suitable as crosslinkers are acrylamides, methacrylamides and N-allylamines of at least difunctional amines. Such amines, are, for example, 1,2-diaminomethane, 1,2-diaminoethane, 1,3-diaminopropane, 1,4-diaminobutane, 1,6-diaminohexane, 1,12-dodecanediamine, piperazine, diethylenetriamine or isophoronediamine. Also suitable are the amides of allylamine and unsaturated carboxylic acids, such as acrylic acid, methacrylic acid, itaconic acid, maleic acid, or at least dibasic carboxylic acids, as described above.

Also suitable as crosslinkers are triallylamine and triallylmonoalkylammonium salts, e.g. triallylmethylammonium chloride or methyl sulfate.

Also suitable are N-vinyl compounds of urea derivatives, at least difunctional amides, cyanurates or urethanes, for example of urea, ethyleneurea, propyleneurea or tartramide, e.g. N,N′-divinylethyleneurea or N,N′-divinylpropyleneurea.

Further suitable crosslinkers are divinyldioxane, tetraallylsilane or tetravinylsilane.

It is of course also possible to use mixtures of the abovementioned compounds. Preference is given to using those crosslinkers which are soluble in the monomer mixture.

Particularly preferred crosslinkers are, for example, methylenebisacrylamide, triallylamine and triallylalkylammonium salts, divinylimidazole, pentaerythritol triallyl ether, N,N′-divinylethyleneurea, reaction products of polyhydric alcohols with acrylic acid or methacrylic acid, methacrylic esters and acrylic esters of polyalkylene oxides or polyhydric alcohols which have been reacted with ethylene oxide and/or propylene oxide and/or epichlorohydrin.

Very particularly preferred crosslinkers are pentaerythritol triallyl ether, methylenebisacrylamide, N,N′-divinylethyleneurea, triallylamine and triallylmonoalkylammonium salts, and acrylic esters of glycol, butanediol, trimethylolpropane or glycerol, or acrylic esters of glycol, butanediol, trimethylolpropane or glycerol reacted with ethylene oxide and/or epichlorohydrin.

The monomers (a) to (e) can in each case be used individually or in a mixture with other monomers from the same group.

The copolymerization takes place in the presence of at least one regulator (e). The term regulator (polymerization regulator) is used to describe compounds with high transfer constants. Regulators accelerate chain transfer reactions and thus effect a lowering of the degree of polymerization of the resulting polymers without influencing the net reaction rate.

With the regulators, it is possible to differentiate between mono-, bi- or polyfunctional regulators, depending on the number of functional groups in the molecule which can lead to one or more chain transfer reactions. Suitable regulators are described in detail, for example, by K. C. Berger and G. Brandrup in J. Brandrup, E. H. Immergut, Polymer Handbook, 3rd edition, John Wiley & Sons, New York, 1989, p. II/81-II/141. Suitable regulators are, for example, aldehydes, such as formaldehyde, acetaldehyde, propionaldehyde, n-butyraldehyde, isobutyraldehyde. Other regulators which can be used are: formic acid, its salts or esters, 2,5-diphenyl-1-hexene, ammonium formate, hydroxylammonium sulfate and hydroxylammonium phosphate.

Further suitable regulators are halogen compounds, such as alkyl halides, such as tetrachloromethane, chloroform, bromotrichloromethane, bromoform, allyl bromide, and benzyl compounds, such as benzyl chloride or benzyl bromide. Further suitable regulators are allyl compounds, such as, for example, allyl alcohol, functional allyl ethers, such as allyl ethoxylates, alkyl allyl ethers, or glycerol monoallyl ether.

The regulators used are preferably compounds which contain sulfur in bonded form.

Compounds of this type are, for example, inorganic hydrogensulfites, disulfites and dithionites or organic sulfides, disulfides, polysulfides, sulfoxides, sulfones. The following regulators are mentioned by way of example: di-n-butyl sulfide, di-n-octyl sulfide, diphenyl sulfide, thiodiglycol, ethylthioethanol, diisopropyl disulfide, di-n-butyl disulfide, di-n-hexyl disulfide, diacetyl disulfide, diethanol sulfide, di-t-butyl trisulfide, dimethyl sulfoxide, dialkyl sulfide, dialkyl disulfide and/or diaryl sulfide.

Particular preference is given to organic compounds which contain sulfur in bonded form.

Preferably, compounds used as polymerization regulators are thiols (compounds which contain sulfur in the form of SH groups, also referred to as mercaptans). Preferred regulators are mono-, bi- and polyfunctional mercaptans, mercaptoalcohols and/or mercaptocarboxylic acids.

Examples of these compounds are allyl thioglycolates, ethyl thioglycolate, cysteine, 2-mercaptoethanol, 1,3-mercaptopropanol, 3-mercaptopropane-1,2-diol, 1,4-mercaptobutanol, mercaptoacetic acid, 3-mercaptopropionic acid, mercaptosuccinic acid, thioglycerol, thioacetic acid, thiourea and alkyl mercaptans, such as n-butyl mercaptan, n-hexyl mercaptan or n-dodecyl mercaptan.

Particularly preferred thiols are cysteine, 2-mercaptoethanol, 1,3-mercaptopropanol, 3-mercaptopropane-1,2-diol, thioglycerol, thiourea.

Examples of bifunctional regulators which contain two sulfurs in bonded form are bifunctional thiols, such as, for example, dimercaptopropanesulfonic acid (sodium salt), dimercaptosuccinic acid, dimercapto-1-propanol, dimercaptoethane, dimercaptopropane, dimercaptobutane, dimercaptopentane, dimercaptohexane, ethylene glycol bis-thioglycolates and butanediol bis-thioglycolate.

Examples of polyfunctional regulators are compounds which contain more than two sulfurs in bonded form. Examples thereof are trifunctional and/or tetrafunctional mercaptans.

Preferred trifunctional regulators are trifunctional mercaptans, such as, for example, trimethylolpropane tris(2-mercaptoethanoate), trimethylolpropane tris(3-mercaptopropionate), trimethylolpropane tris(4-mercaptobutanoate), trimethylolpropane tris(5-mercaptopentanoate), trimethylolpropane tris(6-mercaptohexanoate), trimethylolpropane tris(2-mercaptoacetate).

Glyceryl thioglycolate, glyceryl thiopropionate, glyceryl thioethoxide, glyceryl thiobutanoate, 1,1,1-propanetriyl tris(mercaptoacetate), 1,1,1-propanetriyl tris(mercaptoethanoate), 1,1,1-propanetriyl tris(mercaptopropionate), 1,1,1-propanetriyl tris(mercaptobutanoate), 2-hydroxymethyl-2-methyl-1,3-propanediol tris(mercaptoacetate), 2-hydroxymethyl-2-methyl-1,3-propanediol tris(mercaptoethanoate), 2-hydroxymethyl-2-methyl-1,3-propanediol tris(mercaptopropionate), 2-hydroxymethyl-2-methyl-1,3-propanediol tris(mercaptobutanoate).

Particularly preferred trifunctional regulators are glyceryl thioglycolate, trimethylolpropane tris(2-mercaptoacetate, 2-hydroxymethyl-2-methyl-1,3-propanediol tris(mercaptoacetate).

Preferred tetrafunctional mercaptans are pentaerythritol tetraquis(2-mercaptoacetate), pentaerythritol tetraquis(2-mercaptoethanoate), pentaerythritol tetraquis(3-mercaptopropionate), pentaerythritol tetraquis(4-mercaptobutanoate), pentaerythritol tetraquis(5-mercaptopentanoate), pentaerythritol tetraquis(6-mercaptohexanoate).

Further suitable polyfunctional regulators are Si compounds which are formed by reacting compounds of the formula (IVa). Also suitable as polyfunctional regulators are Si compounds of the formula (IVb). 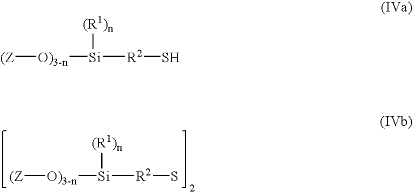
in which
n is a value from 0 to 2,
R¹ is a C₁-C₁₆-alkyl group or phenyl group
R² is a C₁-C₁₈-alkyl group, the cyclohexyl group or phenyl group,
Z is a C₁-C₁₈ alkyl group, C₂-C₁₈-alkylene group or C₂-C₁₈-alkynyl group whose carbon atoms may be replaced by nonadjacent oxygen or halogen atoms, or is one of the groups 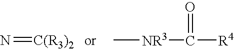
   in which
R₃ is a C₁-C₁₂-alkyl group and
R₄ is a C₁-C₁₈-alkyl group.

Particular preference is given to the compounds IVa, including, primarily, mercaptopropyltrimethoxysilane and mercaptopropyltriethoxysilane.

All of the regulators mentioned can be used individually or in combination with one another.

In a preferred embodiment, 0.1 to 5 parts by weight, preferably 0.2 to 2 parts by weight, and in particular 0.25 to 1 part by weight, of a polymerization regulator (e) is used per 1 part by weight of a crosslinker (monomer (d)).

The present invention further provides polymers obtainable by
(i) free-radically initiated copolymerization of monomer mixtures of
   (a) at least one cationic monomer or quaternizable monomer
   (b) optionally at least one water-soluble monomer,
   (c) optionally at least one further free-radically copolymerizable monomer
   (d) at least one crosslinking monomer having at least two ethylenically unsaturated, nonconjugated double bonds, and
   (e) at least one polyfunctional regulator
(ii) subsequent quaternization or protonation of the polymer if the monomer (a) used is a nonquaternized monomer or an only partially quaternized monomer.

The present invention further provides polymers obtainable by
(i) free-radically initiated copolymerization of monomer mixtures of
   (a) at least one cationic monomer or quaternizable monomer chosen from the group consisting of diallylamines of the formula (II) in which R⁴ is C₁-C₂₄-alkyl 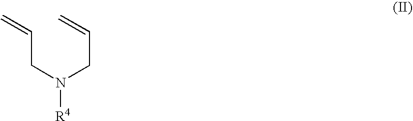
   and N,N-dialkylaminoalkyl acrylates and methacrylates and N,N-dialkylaminoalkylacrylamides and -methacrylamides of the formula (III), 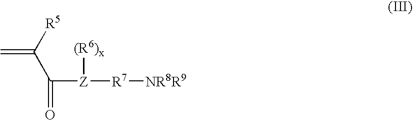
   where R⁵, R⁶, independently, are a hydrogen atom or a methyl radical, R⁷ is an alkylene radical having 1 to 24 carbon atoms, optionally substituted by alkyl radicals, and R⁸, R⁹ are C₁-C₂₄-alkyl radicals. Z is a nitrogen atom together with x=1 or is an oxygen atom together with x=0,
   (b) optionally at least one water-soluble monomer,
   (c) optionally at least one further free-radically copolymerizable monomer,
   (d) at least one crosslinking monomer having at least two ethylenically unsaturated, nonconjugated double bonds, and
   (e) at least one regulator
(ii) subsequent quaternization or protonation of the polymer if the monomer (a) used is a nonquaternized monomer or an only partially quaternized monomer.

WO 93/25592 describes crosslinked polymers of quaternized aminoalkylacrylates or aminoalkylacrylamides with vinyllactams. These polymers are prepared without regulators.

The invention further provides a process for the preparation of polymers by free-radically initiated copolymerization of a monomer mixture of
(a) at least one cationic monomer or quaternizable monomer
(b) optionally at least one water-soluble monomer,
(c) optionally at least one further free-radically copolymerizable monomer
(d) at least one crosslinking monomer having at least two ethylenically unsaturated, nonconjugated double bonds,
   in the presence of a polyfunctional regulator (e)
   and subsequent quaternization or protonation of the polymer if the monomer (a) used is a nonquaternized monomer or an only partially quaternized monomer.

The invention further provides a process for the preparation of polymers by free-radically initiated copolymerization of a monomer mixture of
(a) at least one cationic monomer or quaternizable monomer chosen from the group consisting of diallylamines of the formula (II) in which R⁴ is C₁-C₂₄-alkyl 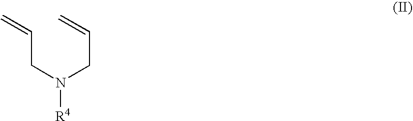
and N,N-dialkylaminoalkyl acrylates and methacrylates and N,N-dialkylaminoalkylacrylamides and -methacrylamides of the formula (III), 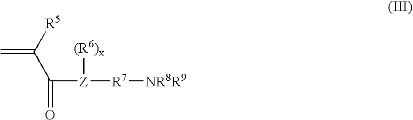
where R⁵, R⁶, independently, are a hydrogen atom or a methyl radical, R⁷ is an alkylene radical having 1 to 24 carbon atoms, optionally substituted by alkyl radicals, and R⁸, R⁹ are C₁-C₂₄-alkyl radicals. Z is a nitrogen atom together with x=1 or is an oxygen atom together with x=0,
(b) optionally at least one water-soluble monomer,
(c) optionally at least one further free-radically copolymerizable monomer,
(d) at least one crosslinking monomer having at least two ethylenically unsaturated, nonconjugated double bonds,
   in the presence of a regulator (e)
   and subsequent quaternization or protonation of the polymer, if the monomer (a) is a nonquaternized monomer or an only partially quaternized monomer.

The polymers can be prepared by processes of free-radically initiated polymerization known per se, e.g. by solution polymerization, emulsion polymerization, suspension polymerization, precipitation polymerization, inverse suspension polymerization or inverse emulsion polymerization, without the methods which can be used being limited thereto.

The monomers (a), (d), (e) and optionally (b) and (c) are copolymerized according to a type of solution polymerization in water and/or polar organic solvents. Suitable polar organic solvents are, for example, water-miscible compounds, such as tetrahydrofuran, N-methylpyrrolidone, dioxane, dimethyl sulfoxide, acetone, glycols, such as ethylene glycol, propylene glycol, 1,4-butanediol, diethylene glycol, triethylene glycol, tetraethylene glycol and block copolymers of ethylene oxide and propylene oxide, and etherified polyalkylene glycols which are obtainable, for example, by alkylation of alkylene glycols and polyalkylene glycols. Suitable compounds are, for example, the glycols or polyethylene glycols containing C₁- to C₄-alkyl end groups. The etherification can be carried out on one side or on both sides. Other suitable solvents are alcohols having 1 to 4 carbon atoms or acetone. It is possible to use either a single solvent, or to carry out the copolymerization also in the presence of solvent mixtures. Particularly preferred solvents are water, C₁- to C₃-alcohols, such as methanol, ethanol, isopropanol and n-propanol, and mixtures of said solvents. The solvents are usually used in an amount such that copolymer solutions with a copolymer content of from 5 to 80% by weight, preferably 10 to 60% by weight, are obtained.

In a preferred embodiment, the regulator (e), optionally in the form of a solution in water and/or a C₁-C₄-alcohol, is metered into the reaction mixture.

In a preferred embodiment, the processes according to the invention are carried out as batch procedures. In this connection, it is preferred to introduce the regulator (e) into the initial charge.

In a preferred embodiment, the processes according to the invention are carried out as feed procedures. In this connection, individual or all of the reactants are added in totality or partially, batchwise or continuously, together or in separate feeds, to a reaction mixture. Thus, for example, a solution of the polymerization regulator and an initiator solution can be added continuously or batchwise to a mixture of the monomers and a solvent at the polymerization temperature within the stated time. It is, however, also possible to meter in a mixture of regulator and initiator into the initial charge heated to the polymerization temperature. Another variant consists in adding the initiator to the initial charge below or at the polymerization temperature and only introducing the regulator or a solution of the regulator into the reaction mixture within a pregiven period after the polymerization temperature has been reached. In a further variant, the initiator and the crosslinker (d) are added to a mixture of regulator (e), monomer (a) and optionally monomers (b) and (c) and a solvent after the polymerization temperature has been reached. It is also possible to heat the initial charge to the polymerization temperature and then to add regulator (e), initiator and monomers (d) in separate feeds or together. It is of course also possible to add regulator (e), initiator, monomers (d) and monomers (a) and optionally monomers (b) and (c) to an initial charge heated to the polymerization temperature. Preference is given to using water or a mixture of water and at least some of the monomers (a) and optionally (b) and (c), and optionally further components as the initial charge. Particular preference here is given to a procedure in which the polymerization regulators (e) are metered in continuously or in portions during the polymerization of the monomers.

The polymerization is usually carried out at temperatures of from 20° C. to 130° C. and at atmospheric pressure or under autogenous pressure.

Initiators which can be used for the free-radical polymerization are the water-soluble and water-insoluble peroxo and/or azo compounds customary for this purpose, for example alkali metal or ammonium peroxydisulfates, dibenzoyl peroxide, tert-butyl perpivalate, tert-butyl-per-2-ethylhexanoate, di-tert-butyl peroxide, tert-butyl hydroperoxide, azobisisobutyronitrile, azobis(2-amidinopropane) dihydrochloride or 2,2′-azobis(2-methylbutyronitrile). Also suitable are initiator mixtures or redox initiator systems, such as, for example, ascorbic acid/iron(II) sulfate/sodium peroxodisulfate, tert-butyl hydroperoxide/sodium disulfite, tert-butyl hydroperoxide/sodium hydroxymethanesulfinate. The initiators can be used in the customary amounts, for example 0.05 to 5% by weight, based on the amount of the monomers to be polymerized.

The molecular weight and the K value of the polymers can be varied within a wide range in a manner known per se through the choice of polymerization conditions, for example polymerization time, polymerization temperature or initiator concentration, and by the content of crosslinker, and regulators.

In a preferred embodiment, the person skilled in the art chooses the reaction conditions such that polymers are formed which have a viscosity below 15 000 mPas (measured in accordance with Brookfield, spindle 4, 12 rpm, 25° C.).

The concentration of the monomers in the reaction medium is usually 5 to 60% by weight and is preferably in the range from 10 to 45% by weight. The polymerization is carried out such that visible gelling of the reaction mixture does not occur. Should gel particles form, these have a diameter of less than 1 mm, preferably less than 500 nm, determined by scattered light measurement in the chosen reaction medium. The resulting copolymers are homogeneously soluble in the reaction medium.

The K values of the polymers are in a range between 10 and 350, preferably 20 and 200 and particularly preferably 35 to 110. The K values are measured in accordance with Fikentscher, Cellulosechemie, vol. 13, pp. 58-64 (1932) at 25° C. at 0.1% strength in 5% by weight sodium chloride solution.

The mixtures formed during the polymerization can, following the polymerization process, be subjected to a physical or chemical after-treatment. Such processes are, for example, the known processes for reducing residual monomers, such as, for example, after-treatment by adding polymerization initiators or mixtures of two or more polymerization initiators at suitable temperatures or heating the polymerization solution to temperatures above the polymerization temperature, an after-treatment of the polymer solution using steam or stripping with nitrogen or treatment of the reaction mixture with oxidizing or reducing reagents, adsorption processes, such as the adsorption of impurities onto selected media, such as, for example, activated carbon, or ultrafiltration. It is also possible to follow with known work-up steps, for example suitable drying processes, such as spray, freeze or roller drying or agglomeration processes following the drying. The mixtures with a low content of residual monomer obtained by the process according to the invention can also be sold directly.

The polymers according to the invention can advantageously be used in cosmetic preparations, in particular hair cosmetic preparations.

The term “cosmetic preparations” is understood broadly and means all those preparations which are suitable for application to skin and/or hair and/or nails and pursue a purpose other than a purely medicinal-therapeutic purpose.

The polymers according to the invention can be used in skin cosmetic preparations.

For example, the polymers according to the invention are used in cosmetic compositions for cleansing the skin. Such cosmetic cleansers are chosen from bar soaps, such as toilet soaps, curd soaps, transparent soaps, luxury soaps, deodorant soaps, cream soaps, baby soaps, skin protection soaps, abrasive soaps and syndets, liquid soaps, such as pasty soaps, soft soaps and washing pastes, and liquid washing, shower and bath preparations, such as washing lotions, shower preparations and gels, foam baths, oil baths and scrub preparations.

The polymers according to the invention are preferably used in cosmetic compositions for care and protection of the skin, in nail care compositions and in preparations for decorative cosmetics.

Particular preference is given to the use in skin care compositions, intimate care compositions, foot care compositions, deodorants, sunscreen compositions, repellents, shaving compositions, hair-removal compositions, antiacne compositions, make-up, mascara, lipsticks, eyeshadows, kohl pencils, eyeliners, blushers, powders and eyebrow pencils.

The skin care compositions are, in particular, in the form of W/O or O/W skin creams, day and night creams, eye creams, face creams, antiwrinkle creams, moisturizing creams, bleaching creams, vitamin creams, skin lotions, care lotions and moisturizing lotions.

In the cosmetic preparations, the polymers according to the invention can develop particular effects. The polymers can, inter alia, contribute to the moistening and conditioning of the skin and to improving the feel of the skin. The polymers can also act as thickeners in the formulations. By adding the polymers according to the invention, it is possible to achieve a considerable improvement in the skin compatibility in certain formulations.

The copolymers according to the invention are present in the skin cosmetic preparations in a proportion of about 0.001 to 20% by weight, preferably 0.01 to 10% by weight, very particularly preferably 0.1 to 5% by weight, based on the total weight of the composition.

Depending on the field of use, the compositions according to the invention can be applied in a form suitable for skin care, such as, for example, in the form of a cream, foam, gel, stick, powder, mousse, milk or lotion.

In addition to the polymers according to the invention and suitable solvents, the skin cosmetic preparations may also comprise additives customary in cosmetics, such as emulsifiers, preservatives, perfume oils, cosmetic active ingredients, such as phytantriol, vitamins A, E and C, retinol, bisabolol, panthenol, sunscreens, bleaches, colorants, tints, tanning agents (e.g. dihydroxyacetone), collagen, protein hydrolyzates, stabilizers, pH regulators, dyes, salts, thickeners, gel formers, bodying agents, silicones, humectants, refatting agents and further customary additives.

Suitable solvents are, in particular, water and lower monoalcohols or polyols having 1 to 6 carbon atoms or mixtures thereof; preferred monoalcohols or polyols are ethanol, isopropanol, propylene glycol, glycerol and sorbitol.

Further customary additives which may be present are fatty bodies, such as mineral and synthetic oils, such as, for example, paraffins, silicone oils and aliphatic hydrocarbons with more than 8 carbon atoms, animal and vegetable oils, such as, for example, sunflower oil, coconut oil, avocado oil, olive oil, lanolin, or waxes, fatty acids, fatty acid esters, such as, for example, triglycerides of C₆-C₃₀-fatty acids, wax esters, such as, for example, jojoba oil, fatty alcohols, vaseline, hydrogenated lanolin and acetylated lanolin. It is of course also possible to use mixtures thereof.

Customary thickeners in such formulations are crosslinked polyacrylic acids and derivatives thereof, polysaccharides, such as xanthan gum, agar agar, alginates or Tyloses, carboxymethylcellulose or hydroxycarboxymethylcellulose, fatty alcohols, monoglycerides and fatty acids, polyvinyl alcohol and polyvinylpyrrolidone.

The polymers according to the invention can also be mixed with conventional polymers if specific properties are to be set.

Suitable conventional polymers are, for example, anionic, cationic, amphoteric and neutral polymers.

Examples of anionic polymers are homo- and copolymers of acrylic acid and methacrylic acid or salts thereof, copolymers of acrylic acid and acrylamide and salts thereof; sodium salts of polyhydroxycarboxylic acids, water-soluble or water-dispersible polyesters, polyurethanes and polyureas. Particularly suitable polymers are copolymers of t-butyl acrylate, ethyl acrylate, methacrylic acid (e.g. Luvimer® 100P), copolymers of ethyl acrylate and methacrylic acid (e.g. Luvimer® MAE), copolymers of N-tert-butylacrylamide, ethyl acrylate, acrylic acid (Ultrahold® 8, strong), copolymers of vinyl acetate, crotonic acid and optionally further vinyl esters (e.g. Luviset® products), maleic anhydride copolymers, optionally reacted with alcohols, anionic polysiloxanes, e.g. carboxyfunctional, copolymers of vinylpyrrolidone, t-butyl acrylate, methacrylic acid (e.g Luviskol® VBM), copolymers of acrylic acid and methacrylic acid with hydrophobic monomers, such as, for example C₄-C₃₀-alkyl esters of meth(acrylic acid), C₄-C₃₀-alkyl vinyl esters, C₄-C₃₀-alkyl vinyl ethers and hyaluronic acid.

Further suitable polymers are cationic polymers with the INCI name Polyquaternium, e.g. copolymers of vinylpyrrolidone/N-vinylimidazolium salts (Luviquat® FC, Luviquat® HM, Luviquat® MS, Luviquat® Care), copolymers of N-vinylpyrrolidone/dimethylaminoethyl methacrylate, quaternized with diethyl sulfate (Luviquat® PQ 11), copolymers of N-vinylcaprolactam/N-vinylpyrrolidone/N-vinylimidazolium salts (Luviquat® Hold); cationic cellulose derivatives (Polyquaternium-4 and -10), acrylamide copolymers (Polyquaternium-7) and chitosan.

Also suitable as further polymers are neutral polymers, such as polyvinylpyrrolidones, copolymers of N-vinylpyrrolidone and vinyl acetate and/or vinyl propionate, polysiloxanes, polyvinylcaprolactam and copolymers with N-vinylpyrrolidone, polyethyleneimines and salts thereof, polyvinylamines and salts thereof, cellulose derivatives, polyaspartic acid salts and derivatives.

To establish certain properties, the preparations can additionally also comprise conditioning substances based on silicone compounds. Suitable silicone compounds are, for example, polyalkylsiloxanes, polyarylsiloxanes, polyarylalkylsiloxanes, polyether siloxanes or silicone resins.

The copolymers according to the invention are used in cosmetic preparations, the preparation of which is carried out in accordance with rules familiar to the person skilled in the art.

Such formulations are advantageously in the form of emulsions, preferably in the form of water-in-oil (W/O) or oil-in-water (O/W) emulsions. However, according to the invention, it is also possible and in some instances advantageous to choose other types of formulation, for example hydrodispersions, gels, oils, oleogels, multiple emulsions, for example in the form of W/O/W or O/W/O emulsions, anhydrous ointments or ointment bases etc.

The emulsions which can be used according to the invention are prepared by known methods.

In addition to the copolymer according to the invention, the emulsions comprise customary constituents, such as fatty alcohols, fatty acid esters and, in particular, fatty acid triglycerides, fatty acids, lanolin and derivatives thereof, natural or synthetic oils or waxes and emulsifiers in the presence of water.

The choice of emulsion type-specific additives and the preparation of suitable emulsions is described, for example, in Schrader, Grundlagen und Rezepturen der Kosmetika [Fundamentals and Formulations of Cosmetics], Hüthig Buch Verlag, Heidelberg, 2nd edition, 1989, third part, to which reference is expressly made here.

Thus, a skin cream which can be used according to the invention can, for example, be in the form of a W/O emulsion. Such an emulsion comprises an aqueous phase which is emulsified in an oil or fatty phase by means of a suitable emulsifier system.

The concentration of the emulsifier system in this type of emulsion is about 4 to 35% by weight, based on the total weight of the emulsion; the fatty phase constitutes about 20 to 60% by weight and the aqueous phase about 20 to 70% by weight, in each case based on the total weight of the emulsion. The emulsifiers are those which are usually used in this type of emulsion. They are, for example, chosen from: C₁₂-C₁₈-sorbitan fatty acid esters; esters of hydroxystearic acid and C₁₂-C₃₀-fatty alcohols; mono- and diesters of C₁₂-C₁₈-fatty acids and glycerol or polyglycerol; condensates of ethylene oxide and propylene glycols; oxypropylenated/oxyethylenated C₁₂-C₂₀-fatty alcohols; polycyclic alcohols, such as sterols; aliphatic alcohols with a high molecular weight, such as lanolin; mixtures of oxypropylenated/polyglycerolated alcohols and magnesium isostearate; succinic esters of polyoxyethylenated or polyoxypropylenated fatty alcohols; and mixtures of magnesium lanolate, calcium lanolate, lithium lanolate, zinc lanolate or aluminum lanolate and hydrogenated lanolin or lanolin alcohol.

Suitable fatty components which may be present in the fatty phase of the emulsions include hydrocarbon oils, such as paraffin oil, purcellin oil, perhydrosqualene and solutions of microcrystalline waxes in these oils; animal or vegetable oils, such as sweet almond oil, avocado oil, calophylum oil, lanolin and derivatives thereof, castor oil, sesame oil, olive oil, jojoba oil, karite oil, hoplostethus oil; mineral oils whose distillation start under atmospheric pressure is at about 250° C. and whose distillation end point is at 410° C., such as, for example, vaseline oil; esters of saturated or unsaturated fatty acids, such as alkyl myristates, e.g. isopropyl myristate, butyl myristate or cetyl myristate, hexadecyl stearate, ethyl palmitate or isopropyl palmitate, octanoic or decanoic acid triglycerides and cetyl ricinoleate.

The fatty phase can also comprise silicone oils soluble in other oils, such as dimethylpolysiloxane, methylphenylpolysiloxane and the silicone-glycol copolymer, fatty acids and fatty alcohols.

In order to favor the retention of oils, it is also possible to use waxes, such as, for example, carnauba wax, candellila wax, beeswax, microcrystalline wax, ozokerite wax and Ca, Mg and Al oleates, myristates, linoleates and stearates.

In general, these water-in-oil emulsions are prepared by adding the fatty phase and the emulsifier to the batch container. The container is heated at a temperature of from 70 to 75° C., then the oil-soluble ingredients are added and, with stirring, water is added which has been heated beforehand to the same temperature and in which the water-soluble ingredients have been dissolved beforehand; the mixture is stirred until an emulsion of the desired fineness is obtained, which is then left to cool to room temperature, if necessary with a lesser amount of stirring.

In addition, a care emulsion according to the invention can be in the form of an O/W emulsion. Such an emulsion usually comprises an oil phase, emulsifiers which stabilize the oil phase in the water phase, and an aqueous phase which is usually present in thickened form.

The aqueous phase of the O/W emulsion of the preparations according to the invention optionally comprises
alcohols, diols or polyols and ethers thereof, preferably ethanol, isopropanol, propylene glycol, glycerol, ethylene glycol monoethyl ether;
customary thickeners or gel formers, such as, for example, crosslinked polyacrylic acids and derivatives thereof, polysaccharides, such as xanthan gum, or alginates, carboxymethylcellulose or hydroxycarboxymethylcellulose, fatty alcohols, polyvinyl alcohol and polyvinylpyrrolidone.

The oil phase comprises oil components customary in cosmetics, such as, for example:
esters of saturated and/or unsaturated, branched and/or unbranched C₃-C₃₀-alkanecarboxylic acids and saturated and/or unsaturated, branched and/or unbranched C₃-C₃₀-alcohols, of aromatic carboxylic acids and saturated and/or unsaturated, branched and/or unbranched C₃-C₃₀-alcohols, for example isopropyl myristate, isopropyl stearate, hexyldecyl stearate, oleyl oleate; and also synthetic, semisynthetic and natural mixtures of such esters, such as jojoba oil;
branched and/or unbranched hydrocarbons and hydrocarbon waxes;
silicone oils, such as cyclomethicone, dimethylpolysiloxane, diethylpolysiloxane, octamethylcyclotetrasiloxane, and mixtures thereof;
dialkyl ethers;
mineral oils and mineral waxes;
triglycerides of saturated and/or unsaturated, branched and/or unbranched C₈-C₂₄-alkanecarboxylic acids; they can be chosen from synthetic, semisynthetic or natural oils, such as olive oil, palm oil, almond oil or mixtures.

Suitable emulsifiers are preferably O/W emulsifiers, such as polyglycerol esters, sorbitan esters or partially esterified glycerides.

The preparation can take place by melting the oil phase at about 80° C.; the water-soluble constituents are dissolved in hot water, and added to the oil phase slowly and with stirring; the mixture is then homogenized and stirred until cold.

The polymers according to the invention are also suitable for use in washing and shower gel formulations and bath preparations.

In addition to the polymers according to the invention, such formulations usually comprise anionic surfactants as base surfactants and amphoteric and nonionic surfactants as cosurfactants, and also lipids, perfume oils, dyes, organic acids, preservatives and antioxidants, and also thickeners/gelling agents, skin conditioning agents and humectants.

All anionic, neutral, amphoteric or cationic surfactants used customarily in body-cleansing compositions can be used in the washing, shower and bath preparations.

The formulations comprise 2 to 50% by weight of surfactants, preferably 5 to 40% by weight, particularly preferably 8 to 30% by weight.

Suitable anionic surfactants are, for example, alkyl sulfates, alkyl ether sulfates, alkylsulfonates, alkylarylsulfonates, alkyl succinates, alkyl sulfosuccinates, N-alkoyl sarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 to 10 ethylene oxide or propylene oxide units, preferably 1 to 3 ethylene oxide units, in the molecule.

Suitable are, for example, sodium lauryl sulfate, ammonium lauryl sulfate, sodium lauryl ether sulfate, ammonium lauryl ether sulfate, sodium lauryl sarcosinate, sodium oleyl succinate, ammonium lauryl sulfosuccinate, sodium dodecylbenzenesulfonate, triethanolamine dodecylbenzenesulfonate.

Suitable amphoteric surfactants are, for example, alkylbetaines, alkylamidopropylbetaines, alkylsulfobetaines, alkyl glycinates, alkyl carboxyglycinates, alkyl amphoacetates or -propionates, alkyl amphodiacetates or -dipropionates.

For example, cocodimethylsulfopropylbetaine, laurylbetaine, cocamidopropylbetaine or sodium cocamphopropionate can be used.

Suitable nonionic surfactants are, for example, the reaction products of aliphatic alcohols or alkylphenols having 6 to 20 carbon atoms in the alkyl chain, which may be linear or branched, with ethylene oxide and/or propylene oxide. The amount of alkylene oxide is about 6 to 60 mol per mole of alcohol. Also suitable are alkylamine oxides, mono- or dialkyl alkanolamides, fatty acid esters of polyethylene glycols, ethoxylated fatty acid amides, alkyl polyglycosides or sorbitan ether esters.

In addition, the washing, shower and bath preparations can comprise customary cationic surfactants, such as, for example, quaternary ammonium compounds, for example cetyltrimethylammonium chloride.

In addition, further customary cationic polymers can also be used, such as, for example, copolymers of acrylamide and dimethyldiallylammonium chloride (Polyquaternium-7), cationic cellulose derivatives (Polyquaternium-4, -10), guar hydroxypropyltrimethylammonium chloride (INCI: Hydroxypropyl Guar Hydroxypropyltrimonium Chloride), copolymers of N-vinylpyrrolidone and quaternized N-vinylimidazole (Polyquaternium-16, -44, -46), copolymers of N-vinypyrrolidone/dimethylaminoethyl methacrylate, quaternized with diethyl sulfate (Polyquaternium-11) and others.

In addition, the washing and shower gel formulations and bath preparations can comprise thickeners, such as, for example, sodium chloride, PEG-55, propylene glycol oleate, PEG-120 methyl glucose dioleate and others, and preservatives, further active ingredients and auxiliaries and water.

Hair cosmetic preparations include, in particular styling agents and/or conditioning agents in hair cosmetic preparations, such as hair treatments, hair mousses, (hair) gels or hair sprays, hair lotions, hair rinses, hair shampoos, hair emulsions, split-end fluids, neutralizing agents for permanent waves, hair colorants and bleaches, hot-oil treatment preparations, conditioners, setting lotions or hair sprays. Depending on the field of use, the hair cosmetic preparations can be applied in the form of an (aerosol) spray, (aerosol) mousse, gel, gelspray, cream, lotion or wax.

The hair cosmetic formulations according to the invention comprise, in a preferred embodiment,
a) 0.05 to 20% by weight of the polymer according to the invention
b) 20 to 99.95% by weight of water and/or alcohol
c) 0 to 79.5% by weight of further constituents

Alcohol is understood as meaning all alcohols customary in cosmetics, e.g. ethanol, isopropanol, n-propanol.

Further constituents are understood as meaning the additives customary in cosmetics, for example propellants, defoamers, interface-active compounds, i.e. surfactants, emulsifiers, foaming agents and solubilizers. The interface-active compounds used may be anionic, cationic, amphoteric or neutral. Further customary constituents may also be, for example, preservatives, perfume oils, opacifiers, active ingredients, UV filters, care substances, such as panthenol, collagen, vitamins, protein hydrolyzates, alpha- and beta-hydroxycarboxylic acids, stabilizers, pH regulators, dyes, viscosity regulators, gelling agents, dyes, salts, humectants, refatting agents, complexing agents and further customary additives.

In addition, these include all styling and conditioning polymers known in cosmetics which can be used in combination with the polymers according to the invention if very specific properties are to be set.

Suitable conventional hair cosmetic polymers are, for example, anionic polymers. Such anionic polymers are homo- and copolymers of acrylic acid and methacrylic acid or salts thereof, copolymers of acrylic acid and acrylamide and salts thereof; sodium salts of polyhydroxycarboxylic acids, water-soluble or water-dispersible polyesters, polyurethanes (Luviset® P.U.R.) and polyureas. Particularly suitable polymers are copolymers of t-butyl acrylate, ethyl acrylate, methacrylic acid (e.g. Luvimer® 100P), copolymers of N-tert-butylacrylamide, ethyl acrylate, acrylic acid (Ultrahold® 8, Strong), copolymers of vinyl acetate, crotonic acid and optionally further vinyl esters (e.g. Luviset® grades), maleic anhydride copolymers, optionally reacted with alcohols, anionic polysiloxanes, e.g. carboxyfunctional copolymers of vinylpyrrolidone, t-butyl acrylate, methacrylic acid (e.g Luviskol® VBM).

In addition, the group of polymers suitable for combining with the polymers according to the invention includes, for example, Balance® CR (National Starch; acrylate copolymer), Balance® 0/55 (National Starch; acrylate copolymer), Balance® 47 (National Starch; octylacrylamide/acrylate/butylaminoethyl methacrylate copolymer), Aquaflex® FX 64 (ISP; isobutylene/ethylmaleimide/hydroxyethylmaleimide copolymer), Aquaflex® SF-40 (ISP/National Starch; VP/vinylcaprolactam/DMAPA acrylate copolymer), Allianz® LT-120 (ISP/Rohm & Haas; acrylate/C1-2 succinate/hydroxyacrylate copolymer), Aquarez® HS (Eastman; polyester-1), Diaformer® Z-400 (Clariant; methacryloylethylbetaine/methacrylate copolymer), Diaformer® Z-711 (Clariant; methacryloylethyl N-oxide/methacrylate copolymer), Diaformer® Z-712 (Clariant; methacryloylethyl N-oxide/methacrylate copolymer), Omnirez® 2000 (ISP; monoethyl ester of poly(methyl vinyl ether/maleic acid in ethanol), Amphomer® HC (National Starch; acrylate/octylacrylamide copolymer), Amphomer® 28-4910 (National Starch; octylacrylamide/acrylate/butylaminoethyl methacrylate copolymer), Advantage® HC 37 (ISP; terpolymer of vinylcaprolactam/vinylpyrrolidone/dimethylaminoethyl methacrylate), Acudyne® 258 (Rohm & Haas; acrylate/hydroxy ester acrylate copolymer), Luviset® PUR (BASF, polyurethane-1), Luviflex® Silk (BASF), Eastman® AQ48 (Eastman).

Very particularly preferred anionic polymers are acrylates with an acid number greater than or equal to 120 and copolymers of t-butyl acrylate, ethyl acrylate, methacrylic acid.

Further suitable hair cosmetic polymers are cationic polymers with the INCI name Polyquaternium, e.g. copolymers of vinylpyrrolidone/N-vinylimidazolium salts (Luviquat® FC, Luviquat® HM, Luviquat® MS, Luviquat® Care), copolymers of N-vinylpyrrolidone/dimethylaminoethyl methacrylate, quaternized with diethyl sulfate (Luviquat® PQ 11), copolymers of N-vinylcaprolactam N-vinylpyrrolidone/N-vinylimidazolium salts (Luviquat® Hold); cationic cellulose derivatives (Polyquaternium-4 and -10), acrylamide copolymers (Polyquaternium-7).

In addition, cationic guar derivatives, such as guar hydroxypropyltrimonium chloride (INCI), can be used.

Further suitable hair cosmetic polymers are also neutral polymers, such as polyvinylpyrrolidones, copolymers of N-vinylpyrrolidone and vinyl acetate and/or vinyl propionate, polysiloxanes, polyvinylcaprolactam and copolymers with N-vinylpyrrolidone, polyethyleneimines and salts thereof, polyvinylamines and salts thereof, cellulose derivatives, polyaspartic acid salts and derivatives.

To set certain properties, the preparations can additionally also comprise conditioning substances based on silicone compounds. Suitable silicone compounds are, for example, polyalkylsiloxanes, polyarylsiloxanes, polyarylalkylsiloxanes, polyether siloxanes, silicone resins or Dimethicone Copolyols (CTFA) and aminofunctional silicone compounds such as Amodimethicone (CTFA).

The polymers according to the invention are suitable, in particular, as setting agents in hair styling preparations, in particular hair sprays (aerosol sprays and pump sprays without propellant gas) and hair mousses (aerosol mousses and pump mousses without propellant gas).

In a preferred embodiment, these preparations comprise
a) 0.1 to 10% by weight of the polymer according to the invention
b) 20 to 99.9% by weight of water and/or alcohol
c) 0 to 70% by weight of a propellant
d) 0 to 20% by weight of further constituents.

Propellants are the propellants customarily used for hair sprays or aerosol mousses. Preference is given to mixtures of ropane/butane, pentane, dimethyl ether, 1,1-difluoroethane (HFC-152 a), carbon dioxide, nitrogen or compressed air.

A formulation for aerosol hair mousses preferred according to the invention comprises
a) 0.1 to 10% by weight of the polymer according to the invention
b) 55 to 99.8% by weight of water and/or alcohol
c) 5 to 20% by weight of a propellant
d) 0.1 to 5% by weight of an emulsifier
e) 0 to 10% by weight of further constituents

Emulsifiers which can be used are all emulsifiers customarily used in hair mousses. Suitable emulsifiers may be nonionic, cationic or anionic or amphoteric.

Examples of nonionic emulsifiers (INCI nomenclature) are laureths, e.g. laureth-4; ceteths, e.g. ceteth-1, polyethylene glycol cetyl ether; ceteareths, e.g. ceteareth-25, polyglycol fatty acid glycerides, hydroxylated lecithin, lactyl esters of fatty acids, alkyl polyglycosides.

Examples of cationic emulsifiers are cetyldimethyl-2-hydroxyethylammonium dihydrogenphosphate, cetyltrimonium chloride, cetyltrimonium bromide, cocotrimonium methylsulfate, Quaternium-1 to x (INCI).

Anionic emulsifiers can, for example, be chosen from the group of alkylsulfates, alkyl ether sulfates, alkylsulfonates, alkylarylsulfonates, alkylsuccinates, alkylsulfosuccinates, N-alkoyl sarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

A preparation suitable according to the invention for styling gels can, for example, have the following composition:
a) 0.1 to 10% by weight of the polymer according to the invention
b) 60 to 99.85% by weight of water and/or alcohol
c) 0.05 to 10% by weight of a gelling agent
d) 0 to 20% by weight of further constituents

Gelling agents which can be used are all gelling agents customary in cosmetics. These include slightly crosslinked polyacrylic acid, for example Carbomer (INCI), cellulose derivatives, e.g. hydroxypropylcellulose, hydroxyethylcellulose, cationically modified celluloses, polysaccharides, e.g. xanthum gum, caprylic/capric triglycerides, sodium acrylates copolymer, Polyquaternium-32 (and) Paraffinum Liquidum (INCI), Sodium Acrylates Copolymer (and) Paraffinum Liquidum (and) PPG-1 Trideceth-6, Acrylamidopropyl Trimonium Chloride/Acrylamide Copolymer, Steareth-10 Allyl Ether Acrylates Copolymer, Polyquaternium-37 (and) Paraffinum Liquidum (and) PPG-1 Trideceth-6, Polyquaternium 37 (and) Propylene Glycol Dicaprate Dicaprylate (and) PPG-1 Trideceth-6, Polyquaternium-7, Polyquaternium-44.

The polymers according to the invention can also be used in shampoo formulations as setting and/or conditioning agents. Suitable conditioning agents are, in particular, polymers with a cationic charge. Preferred shampoo formulations comprise
a) 0.05 to 10% by weight of the polymer according to the invention
b) 25 to 94.95% by weight of water
c) 5-50% by weight of surfactants
d) 0-5% by weight of a further conditioning agent
e) 0-10% by weight of further cosmetic constituents

All anionic, neutral, amphoteric or cationic surfactants customarily used in shampoos can be used in the shampoo formulations.

Suitable anionic surfactants are, for example, alkyl sulfates, alkyl ether sulfates, alkylsulfonates, alkylarylsulfonates, alkyl succinates, alkylsulfosuccinates, N-alkoylsarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units in the molecule.

Suitable are, for example, sodium lauryl sulfate, ammonium lauryl sulfate, sodium lauryl ether sulfate, ammonium lauryl ether sulfate, sodium lauroyl sarcosinate, sodium oleyl succinate, ammonium lauryl sulfosuccinate, sodium dodecylbenzenesulfonate, triethanolamine dodecylbenzenesulfonate.

Suitable amphoteric surfactants are, for example, alkylbetaines, alkylamidopropylbetaines, alkylsulfobetaines, alkyl glycinates, alkyl carboxyglycinates, alkyl amphoacetates or propionates, alkyl amphodiacetates or dipropionates.

For example, cocodimethylsulfopropylbetaine, laurylbetaine, cocamidopropylbetaine or sodium cocamphopropionate can be used.

Suitable nonionic surfactants are, for example, the reaction products of aliphatic alcohols or alkylphenols having 6 to 20 carbon atoms in the alkyl chain, which may be linear or branched, with ethylene oxide and/or propylene oxide. The amount of alkylene oxide is about 6 to 60 mol per mole of alcohol. In addition, alkylamine oxides, mono- or dialkylalkanolamides, fatty acid esters of polyethylene glycols, alkyl polyglycosides or sorbitan ether esters are suitable.

In addition, the shampoo formulations can comprise customary cationic surfactants, such as, for example, quaternary ammonium compounds, for example cetyltrimethylammonium chloride.

In the shampoo formulations, to achieve certain effects, it is possible to use customary conditioning agents in combination with the polymers according to the invention. These include, for example, cationic polymers with the INCI name Polyquaternium, in particular copolymers of vinylpyrrolidone/N-vinylimidazolium salts (Luviquat® FC, Luviquat® HM, Luviquat® MS, Luviquat® Care), copolymers of N-vinylpyrrolidone/dimethylaminoethyl methacrylate, quaternized with diethyl sulfate (Luviquat® PQ 11), copolymers of N-vinylcaprolactam/N-vinylpyrrolidone/N-vinylimidazolium salts (Luviquat® Hold); cationic cellulose derivatives (Polyquaternium-4 and -10), acrylamide copolymers (Polyquaternium-7). It is also possible to use protein hydrolyzates, and conditioning substances based on silicone compounds, for example polyalkylsiloxanes, polyarylsiloxanes, polyarylalkylsiloxanes, polyethersiloxanes or silicone resins. Further suitable silicone compounds are Dimethicone Copolyols (CTFA) and aminofunctional silicone compounds, such as Amodimethicone (CTFA). In addition, cationic guar derivatives, such as guar hydroxypropyltrimonium chloride (INCI) can be used.

TABLE 2

| | X | Y | Y | | | | | | | |
|---|---|---|---|---|---|---|---|---|---|---|
| | TAA | MCOH | PE(MC)₄ | k value | Solids | Tel Quel | Reduction in combing force | | | |
| | Monomer | Monomer | Monomer | 5% NaCl | content | viscosity | Wet | | Dry | |
| Feed | (d) | (e) | (e) | solution | (%) | (mPas) | (grade) | (%) | (grade) | (%) |
| 1 | 0.75 | 0.3 | — | 73.4 | 10 | 450 | 1-2 | 53% | 1-2 | 72% |
| 2 | 0.75 | 0.3 | — | 77.8 | 12 | 12800 | 1-2 | 51% | 1-2 | 80% |
| 3 | 0.75 | — | 0.3 | 88.7 | 10 | 3700 | 1-2 | 49% | 1-2 | 83% |
| C1 | 0.75 | — | — | n.m. | 12 | >50000 | 1-2 | 52% | 1-2 | 74% |
| C2 | — | 0.3 | — | 24 | 13 | 150 | 2− | 13% | 2+ | 34% |
| C3 | 0.75 | — | — | 83 | 7 | 3200 | 1-2 | 50% | 1-2 | 75% |

Abbreviations used (% by weight, in each case based on the total amount of monomer)
TAA = triallylamine (% by weight)
MCOH = mercaptoethanol (% by weight)
PE(MC)₄ = pentaerythritol tetramercaptoacetate (% by weight)
n.m. = not measurable due to the excessive amount of gel particles in the solution
The viscosity was determined using a Brookfield viscosimeter with spindle 4, 12 rpm at 25° C.

### EXAMPLES

A: Preparation of Polymers 1 to 3

85.9 g of N-vinylpyrrolidone, 47.7 g of 3-methyl-1-vinylimidazolium methylsulfate (45% in water), X g of triallylamine (amount in Table 1) Y g of regulator (amount and regulator in Table 1) and varying amounts of water (750 to 920 g depending on the solids content; solids content data in Table 1) were initially introduced into a stirred apparatus and heated to 65° C. in a stream of nitrogen with stirring. Then 25 g of a feed consisting of 1.6 g of 2,2′-azobis(2-amidinopropane) dihydrochloride in 65 g of water were metered in over 3 hours. When the metered addition was complete, the mixture was stirred for a further hour at 65° C. The polymerization temperature was then increased to 70° C., and the remaining 2,2′-azobis(2-amidinopropane) dihydrochloride feed was added over a further hour. At the end of this metered addition, the mixture was stirred for a further 2 hours.

This gave colorless polymer solutions with varying solids contents and viscosity (data in Table 2).

The comparison experiments were carried out accordingly with the components specified in Table 1.

B: Comparison of the Properties of the Polymers According to the Invention with the Polymers of the Comparative Examples

For the performance investigation, the polymers were used in a surfactant solution formulation having the following composition:
40.0% of Texapon NSO (sodium laureth sulfate solution 28%; Cognis)
10.0% of Tego-Betaine L7 (cocamidopropylbetaine solution 30%; Goldschmidt)
0.5% of polymer (solids content) ad 100% of water
   Determination of the Combability

The following instructions describe the procedure for determining the wet and dry combability of hair following treatment with conditioning agents. All measurements were carried out in a climatically controlled room at 65% relative humidity and 21° C.

Instruments Used

Wet combability: Frank tensile/force tester
Dry combability: Diastron force measuring system
Digital balance: (top-pan balance)
   Hair:
a) European, bleached: hair tresses from Wernesgrün (bleaching see below)
b) Asiatic, untreated: hair tresses from Wernesgrün with split ends

The following tests were carried out:
Wet combability following application of shampoo on European, bleached hair
Dry combability following application of shampoo on Asiatic hair
   Pretreatment/Cleansing of the Hair:

Prior to the first use, the Asiatic hair tresses were cleansed in a solvent mixture (ethanol/isopropanol/acetone/water 1:1:1:1) until the hair appeared clean in the dry state (i.e. no longer stuck together). The hair was then washed with sodium lauryl ether sulfate.

The European hair was then treated with a bleaching paste (7.00 g of ammonium carbonate, 8.00 g of calcium carbonate, 0.50 g of Aerosil 200, 9.80 g of hydrogen peroxide (30% strength), 9.80 g of demineralized water). The hair tresses were completely immersed into the bleaching paste to ensure extensive wetting of the entire surface of the hair. The tresses were then wiped between the fingers in order to remove excess bleaching paste. The contact time of the remaining bleach on the hair is matched to the degree of damage required, and is generally 15 to 30 minutes, but can vary depending on the quality of the hair. The bleached hair tresses were then thoroughly rinsed under running tap water (2 minutes) and washed with sodium lauryl ether sulfate. The hair was then dipped briefly into an aqueous acidic solution (e.g. citric acid) because of so-called insidious bleaching, and then rinsed with tap water.

Applications:

The hair tress is dipped for 1 minute into the surfactant formulation to be tested, shampooed for 1 minute and then rinsed for 1 minute under running drinking water (hand-hot).

I) Wet Combability

Determination of the blank value for wet combability: the washed hair was dried overnight in a climatically controlled room. Prior to measurement, it was shampooed twice with Texapon NSO for a total of 1 minute and rinsed for 1 minute so that it is definitely wet, i.e. swollen. Prior to the start of the measurement, the tress was precombed until knots were no longer present in the hair and thus a constant application of force is necessary for the repeated measurement combing. The tress was then fixed to a support and combed using the finely-toothed side of the comb into the finely-toothed side of the test comb. The hair was inserted into the test comb for each measurement uniformly and without tension. The measurement was started and evaluated by means of software (EGRANUDO program, Frank). The individual measurement was repeated 5 to 10 times. The calculated average value was noted.

Determination of the measurement value for wet combability: following the determination of the blank value, the hair was treated depending on the desired application. The combing force is measured analogously to the blank-value determination.

Evaluation:
Reduction in combing force wet [%]=100−(measurement value*100/blank value)
II) Dry Combability

Determination of the blank value for dry combability: the washed hair was dried overnight in a climatically controlled room. Prior to the start of the measurement, the tress was precombed until knots were no longer present in the hair and thus a constant application of force is necessary for repeated measurement combing. The tress was then fixed to a support and combed into the finely-toothed side of the test comb. The hair was inserted into the test comb for each measurement uniformly and without tension. The measurement was started and evaluated by means of software (mtt-win, DIASTRON). The individual measurement was repeated 5 to 10 times. The calculated average value was noted together with the standard deviation.

Determination of the measurement value for dry combability: following the determination of the blank value, the hair was treated according to the desired application and dried overnight. The combing force was measured analogously to the blank-value determination. Evaluation:
Decreasing combing force dry [%]=100−(measurement value*100/blank value)

The results are summarized in Table 2. The polymers to be used according to the invention (Examples 1 to 3) exhibit good performance properties and can be prepared with high solids contents.

In contrast to this, polymers which have been prepared without monomer (e) produce excessively high viscosities (C-1) or an excessively low solids content (C-3). Polymers which have been prepared by polymerization in the presence of a regulator but without crosslinker (C-2) exhibit good viscosities, but are unsatisfactory with regard to their performance properties.

The polymers to be used according to the invention produce excellent results with regard to dry combability, but, in particular, to wet combability. A further advantage is that clear (washing) formulations are also possible with the polymer according to the invention.

A further advantage is that the polymers to be used according to the invention can be prepared with high solids contents.

### EXAMPLE 4

In a 1 l stirred apparatus, 400 ml of water, 50 g of N-vinylpyrrolidone and 50 g of dimethyldiallylammonium chloride solution (65% strength) were heated to 80° C. in a stream of nitrogen with stirring using an anchor stirrer at 200 revolutions/min. At this temperature, a first feed comprising 2 g of 2,2′-azobis(2-methylbutyronitrile) and 0.3 g of mercaptoethanol in 30 g of isopropanol and, in parallel, a second feed comprising 2 g of divinylethyleneurea in 30 ml of isopropanol was added over the course of 1.5 hours. The reaction mixture was stirred for a further 3 hours at this temperature.

The isopropyl alcohol was then removed from the reaction mixture by means of steam distillation.

### EXAMPLE 5

In a 1 l stirred apparatus, 400 ml of water, 100 g of dimethyldiallylammonium chloride solution (65% strength) were heated to 80° C. in a stream of nitrogen with stirring using an anchor stirrer at 200 revolutions/min. At this temperature, a first feed comprising 2 g of 2,2′-azobis(2-methylbutyronitrile) and 2.5 g of mercaptoethanol in 30 g of isopropanol and, in parallel, a second feed comprising 8 g of N,N′-divinylethyleneurea in 70 ml of isopropanol was added over the course of 1.9 hours. The reaction mixture was stirred for a further 2 hours at this temperature. The isopropyl alcohol was then removed from the reaction mixture by means of steam distillation.

### EXAMPLE 6

In a 1 l stirred apparatus, 400 ml of water were heated to 80° C. in a stream of nitrogen with stirring using an anchor stirrer at 200 revolutions/min. At this temperature, a first feed comprising 2 g of 2,2′-azobis(2-methylbutyronitrile) and 2 g of mercaptoethanol in 30 g of isopropanol and, in parallel, a second feed comprising 6 g of methylenebisacrylamide, 50 g of N-vinylpyrrolidone and 50 g of 2-methyl-1-vinylimidazolium methylsulfate solution (45% strength) was added over the course of 2 hours. The reaction mixture was stirred for a further 3.5 hours at this temperature. The isopropyl alcohol was then removed from the reaction mixture by means of steam distillation.

### EXAMPLE 7

In a 1 l stirred apparatus, 400 ml of water, 50 g of N-vinylpyrrolidone and 50 g of 2-methyl-1-vinylimidazolium methylsulfate solution (45% strength) were heated to 80° C. in a stream of nitrogen with stirring using an anchor stirrer at 200 revolutions/min. At this temperature, a first feed comprising 2 g of 2,2′-azobis(2-methylbutyronitrile) and 2 g of mercaptoethanol in 30 g of isopropanol and, in parallel, a second feed comprising 4 g of divinylethyleneurea in 30 ml of isopropanol was added over the course of 1.75 hours. The reaction mixture was stirred for a further 3.5 hours at this temperature. The isopropyl alcohol was then removed from the reaction mixture by means of steam distillation.

### EXAMPLE 8

In a 1 l stirred apparatus, 400 ml of water, 50 g of N-vinylpyrrolidone and 50 g of N,N-dimethylaminoethylmethacrylate methyl chloride salt solution (50% strength) were heated to 80° C. in a stream of nitrogen with stirring using an anchor stirrer at 200 revolutions/min. At this temperature, a first feed comprising 2 g of 2,2′-azobis(2-methylbutyronitrile) and 5 g of mercaptoethanol in 30 g of isopropanol and, in parallel, a second feed comprising 10 g of divinylethyleneurea in 70 ml of isopropanol was added over the course of 1.85 hours. The reaction mixture was stirred for a further 3.5 hours at this temperature. The isopropyl alcohol was then removed from the reaction mixture by means of steam distillation.

### EXAMPLE 9

In a 1 l stirred apparatus, 400 ml of water and 100 g of N,N-dimethylaminoethyl methacrylate methyl chloride salt solution (50% strength) were heated to 80° C. in a stream of nitrogen with stirring using an anchor stirrer at 200 revolutions/min. At this temperature, a first feed comprising 2 g of 2,2′-azobis(2-amidinopropane) dihydrochloride and 5 g of mercaptoethanol in 30 g of water and, in parallel, a second feed comprising 8 g of triethylene glycol diacrylate in 50 ml of water was added over the course of 2 hours. The reaction mixture was then stirred for a further 3 hours at this temperature. The reaction mixture was then subjected to steam distillation.

### EXAMPLE 10

In a 1 l stirred apparatus, 400 ml of water and 50 g of vinylpyrrolidbne and 50 g of N,N-dimethylaminoethyl methacrylate methyl chloride salt solution (50% strength) were heated to 80° C. in a stream of nitrogen with stirring using an anchor stirrer at 200 revolutions/min. At this temperature, a first feed comprising 1 g of 2,2′-azobis(2-methylbutyronitrile) and 1 g of mercaptoethanol in 30 g of isopropanol and, in parallel, a second feed comprising 2 g of divinylethyleneurea in 30 ml of isopropanol was added over the course of 1.5 hours. The reaction mixture was stirred for a further 3 hours at this temperature. The isopropanol was then removed from the reaction mixture by means of steam distillation.

### EXAMPLE 11

### Clear Conditioner Shampoo

| % | Ingredient | INCI |
|---|---|---|
| 15.00 | Tego Betaine L 7 | Cocamidopropylbetaine |
| 10.00 | Amphotensid GB 2009 | Disodium cocoamphodiacetate |
| 5.00 | Cremophor PS 20 | Polysorbate 20 |
| 5.00 | Plantacare 2000 | Decyl glucoside |
| 3.00 | Stepan PEG 6000 DS | PEG-150 distearate |
| q.s. | Perfume | |
| q.s. | Preservative | |
| q.s. | Citric acid | |
| 0.1-1.0 | Active substance of conditioner polymer as in | |
| | Example 1 | |
| 2.00 | Rewopal LA 3 | Laureth-3 |
| ad 100 | Water, demineralized | Aqua dem. |

### EXAMPLE 12

### Conditioner Shampoo

| % | Ingredient | INCI |
|---|---|---|
| 30.00 | Texapon NSO | Sodium laureth sulfate |
| 6.00 | Dehyton G | Sodium cocoamphoacetate |
| 6.00 | Dehyton K | Cocamidopropylbetaine |
| 3.00 | Euperlan PK 771 | Sodium laureth sulfate, |
| | | glycol distearate, |
| | | cocamide MEA, laureth-10 |
| 0.1-1.0 | Active substance of conditioner polymer as in Example 1 | |
| 2.00 | Dimethicone | |
| q.s. | Perfume | |
| q.s. | Preservative | |
| q.s. | Citric acid | |
| 1.00 | Sodium chloride | |
| ad 100 | Water, demineralized | Aqua dem. |

### EXAMPLE 13

### Conditioner Shampoo

| % | Ingredient | INCI |
|---|---|---|
| 30.00 | Texapon NSO | Sodium laureth sulfate |
| 6.00 | Dehyton G | Sodium cocoamphoacetate |
| 6.00 | Dehyton K | Cocamidopropylbetaine |
| 3.00 | Euperlan PK 771 | Sodium laureth sulfate, |
| | | glycol distearate, cocamide |
| | | MEA, laureth-10 |
| 0.1-1.0 | Active substance of conditioner polymer as in Example 2 | |
| 2.00 | Amidodimethicone | |
| q.s. | Perfume | |
| q.s. | Preservative | |
| q.s. | Citric acid | |
| 1.00 | Sodium chloride | |
| ad 100 | Water, demineralized | Aqua dem. |

### EXAMPLE 14

### Conditioner Shampoo

| % | Ingredient | INCI |
|---|---|---|
| 40.00 | Texapon NSO | Sodium laureth sulfate |
| 10.00 | Dehyton K | Cocamidopropylbetaine |
| 3.00 | Euperlan PK 771 | Sodium laureth sulfate, |
| | | glycol distearate, cocamide |
| | | MEA, laureth-10 |
| 0.1-1.0 | Active substance of conditioner polymer as in Example 2 | |
| 2.00 | Dow Corning 3052 | |
| q.s. | Perfume | |
| q.s. | Preservative | |
| q.s. | Citric acid | |
| 2.00 | Cocamido DEA | |
| ad 100 | Water, demineralized | Aqua dem. |

### EXAMPLE 15

### Conditioner Shampoo

| % | Ingredient | INCI |
|---|---|---|
| 30.00 | Texapon NSO | Sodium laureth sulfate |
| 6.00 | Dehyton G | Sodium cocoamphoacetate |
| 6.00 | Dehyton K | Cocamidopropylbetaine |
| 3.00 | Euperlan PK 771 | Sodium laureth sulfate, |
| | | glycol distearate, cocamide |
| | | MEA, laureth-10 |
| 0.1-1.0 | Active substance of conditioner polymer as in Example 2 | |
| 2.00 | Dimethicone | |
| q.s. | Perfume | |
| q.s. | Preservative | |
| q.s. | Citric acid | |
| 2.00 | Cocamido DEA | |
| ad 100 | Water, demineralized | Aqua dem. |

### EXAMPLE 16

### Anti-Dandruff Shampoo

| % | Ingredient | INCI |
|---|---|---|
| 40.00 | Texapon NSO | Sodium laureth sulfate |
| 10.00 | Tego Betain L 7 | Cocamidopropylbetaine |
| 10.00 | Rewopol SB FA 30 | Disodium laureth sulfosuccinate |
| 2.50 | Euperlan PK 771 | Sodium laureth sulfate, |
| | | glycol distearate, cocamide |
| | | MEA, laureth-10 |
| 0.1-1.0 | Active substance of conditioner polymer as in Example 3 | |
| 0.50 | Crinipan AD | Climbazole |
| q.s. | Perfume | |
| q.s. | Preservative | |
| q.s. | Citric acid | |
| 0.50 | Sodium chloride | |
| ad 100 | Water, demineralized | Aqua dem. |

### EXAMPLE 17

### Shampoo

| % | Ingredient | INCI |
|---|---|---|
| 25.00 | Sodium laureth sulfate | |
| 5.00 | Cocamidopropylbetaine | |
| 2.50 | Euperlan PK 771 | Sodium laureth sulfate, |
| | | glycol distearate, cocamide |
| | | MEA, laureth-10 |
| 0.1-1.0 | Active substance of conditioner polymer as in Example 3 | |
| 2.0 | Cocamido DEA | |
| q.s. | Perfume | |
| q.s. | Preservative | |
| ad 100 | Water, demineralized | Aqua dem. |

### EXAMPLE 18

### Shampoo

| % | Ingredient | INCI |
|---|---|---|
| 20.00 | Ammonium laureth sulfate | |
| 15.00 | Ammonium lauryl sulfate | |
| 5.00 | Cocamidopropylbetaine | |
| 2.50 | Euperlan PK 771 | Sodium laureth sulfate, |
| | | glycol distearate, cocamide |
| | | MEA, laureth-10 |
| 0.1-1.0 | Active substance of conditioner polymer as in Example 3 | |
| q.s. | Perfume | |
| q.s. | Preservative | |
| 0.50 | Sodium chloride | |
| ad 100 | Water, demineralized | Aqua dem. |

### EXAMPLE 19

### Shampoo

| % | Ingredient | INCI |
|---|---|---|
| 20.00 | Sodium laureth sulfate | |
| 15.00 | Sodium lauryl sulfate | |
| 5.00 | Cocamidopropylbetaine | |
| 2.50 | Euperlan PK 771 | Sodium laureth sulfate, |
| | | glycol distearate, cocamide |
| | | MEA, laureth-10 |
| 0.1-1.0 | Active substance of conditioner polymer as in Example 2 | |
| q.s. | Perfume | |
| q.s. | Preservative | |
| 0.50 | Sodium chloride | |
| ad 100 | Water, demineralized | Aqua dem. |

### EXAMPLE 20

### Clear Shower Gel

| % | Ingredient | INCI |
|---|---|---|
| 40.00 | Texapon NSO | Sodium laureth sulfate |
| 5.00 | Plantacare 2000 | Decyl glucoside |
| 5.00 | Tego Betain L 7 | Cocamidopropylbetaine |
| 0.1-1.0 | Active substance of conditioner | |
| | polymer as in Example 2 | |
| 1.00 | D-Panthenol USP | Panthenol |
| q.s. | Perfume | |
| q.s. | Preservative | |
| q.s. | Citric acid | |
| 2.00 | Sodium chloride | |
| ad 100 | Water, demineralized | Aqua dem. |

### EXAMPLE 21

### Shampoo

| % | Ingredient | INCI |
|---|---|---|
| 12.00 | Texapon N 70 | Sodium laureth sulfate |
| 1.50 | Plantacare 2000 | Decyl glucoside |
| 2.50 | Dehyton PK 45 | Cocamidopropylbetaine |
| 5.00 | Lamesoft PO 65 | Cocoglucoside glyceryl oleate |
| 2.00 | Euperlan PK 771 | Sodium laureth sulfate, |
| | | glycol distearate, cocamide |
| | | MEA, Laureth-10 |
| 0.1-1.0 | Active substance of conditioner polymer as in Example 1 | |
| q.s. | Preservative | |
| q.s. | Sicovit Sunset | Sunset Yellow C.I. 15 985 |
| | Yellow 85 E 110 | |
| q.s. | Perfume | |
| 1.00 | Sodium chloride | |
| ad 100 | Water, demineralized | |

### EXAMPLE 22

### Shampoo

| % | Ingredient | INCI |
|---|---|---|
| 12.00 | Texapon N 70 | Sodium laureth sulfate |
| 1.50 | Plantacare 2000 | Decyl glucoside |
| 2.50 | Dehyton PK 45 | Cocamidopropylbetaine |
| 5.00 | Lamesoft PO 65 | Cocoglucoside glyceryl oleate |
| 2.00 | Euperlan PK 771 | Sodium laureth sulfate, |
| | | glycol distearate, cocamide |
| | | MEA, Laureth-10 |
| 0.1-1.0 | Active substance of conditioner polymer as in Example 1 | |
| q.s. | Preservative | |
| q.s. | Sicovit Sunset | Sunset Yellow C.I. 15 985 |
| | | Yellow 85 E 110 |
| q.s. | Perfume | |
| 1.00 | Sodium chloride | |
| ad 100 | Water, demineralized | |

1. Hair cosmetic preparation comprising polymers obtainable by
(i) free-radically initiated copolymerization of monomer mixtures of
(a) at least one cationic monomer or quaternizable monomer
(b) optionally a water-soluble monomer,
(c) optionally a further free-radically copolymerizable monomer
(d) at least one crosslinking monomer having at least two ethylenically unsaturated, nonconjugated double bonds, and
(e) at least one regulator, where compounds which comprise sulfur in bonded form are used as regulator (e),
(ii) subsequent quaternization or protonation of the polymer if the monomer (a) used is a nonquaternized monomer or an only partially quaternized monomer.

2. Cosmetic preparation containing as conditioning agent polymers obtainable by
(i) free-radically initiated copolymerization of monomer mixtures of
(a) at least one cationic monomer or quarternizable monomer
(b) optionally a water-soluble monomer,
(c) optionally a further free-radically copolymerizable monomer
(d) at least one crosslinking monomer having at least two ethylenically unsaturated, nonconjugated double bonds, and
(e) at least one regulator, where compounds which comprise sulfinur in bonded form are used as regulator (e),
(ii) subsequent quaternization or protonation of the polymer if the monomer (a) used is a nonquaternized momomer or an only partially quaternized monomer.

3. The cosmetic preparation as claimed in claim 2 being a skin and/or hair cosmetic preparation.

4. The hair cosmetic preparation as claimed in claim 1 where N-vinylimidazole derivatives of the formula (I), in which R¹ to R³ are hydrogen, C₁-C₄-alkyl or phenyl, are used as monomer (a)

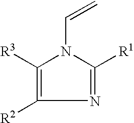

5. The hair cosmetic preparation as claimed in claim 1, where N-vinyllactams are used as monomer (b).

6. The hair cosmetic preparation as claimed in claim 5, where thiols are used as regulator.

7. A polymer obtainable by
(i) free-radically initiated copolymerization of monomer mixtures of
(a) at least one cationic monomer or quaternizable monomer mixtures
(b) optionally at least one water-soluble monomer,
(c) optionally at least one further free-radically copolymerizable monomer
(d) at least one crosslinking monomer having at least two ethylenically unsaturated, nonconjugated double bonds, and
(e) at least one polyfunctional regulator
(ii) subsequent quaternization or protonation of the polymer if the monomer (a) used is a nonquaternized monomer or an only partially quternized monomer.

8. A polymer as claimed in claim 7, where N-vinylimidazole derivatives of the formula (I) in which R¹ to R³ are hydrogen, C₁-C₄-alkyl or phenyl are used as monomer (a).

9. A polymer as claimed in claim 7, where vinyllactams are used as monomer (b).

10. A polymer as claimed in claim 7, where compounds which comprise sulfur in bonded form are used as polyfunctional regulator (e).

11. A polymer as claimed in claim 10, where thiols are used as polyfunctional regulator (e).

12. A polymer as claimed in claim 7 obtainable by
(i) free-radically initiated copolymerization of monomer mixtures of
(a) 1 to 99.98% by weight of at least one cationic monomer or quaternizable monomer
(b) 0 to 98.98% by weight of at least one water-soluble monomer,
(c) 0 to 50% by weight of at least one further free-radically copolymerizable monomer and
(d) 0.01 to 10% by weight of at least one crosslinking monomer having at least two ethylenically unsaturated, nonconjugated double bonds, and
(e) 0.01 to 10% by weight of at least one polyfunctional regulator
(ii) subsequent quaternization or protonation of the polymer if the monomer (a) used is a nonquaternized monomer or an only partially quaternized monomer.

13. A process for the preparation of the polymers by free-radical initiated copolymerization of the monomer mixture of
(a) at least one cationic monomer or quaternizable monomer
(b) optionally at least one water-soluble monomer,
(c) optionally at least one further free-radically copolymerizable monomer
(d) at least one crosslinking monomer having at least two ethylenically unsaturated, nonconjuaged double bonds, in the presence of a polyfincational regulator (e) and subsequent quaternzation or protonation of the polymer if the monomer (a) used is a nonquaternized monomer or an only partially quaternized monomer.

14. A polymer obtainable by
(i) free-radically initiated copolymerization of monomer mixtures of
(a) 2 to 70% by weight of a cationic monomer or quaternizable monomer chosen from the group consisting of diallylamines of the formula (II), in which R⁴ is C₁-C₂₄-alkyl 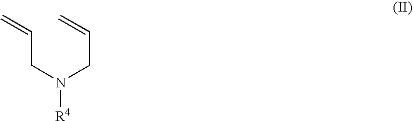
and N,N-dialkylaminoalkyl acrylates and methacrylates and N,N-dialkylaminoalkylacrylamides and-methcrylamides of the formula (III), 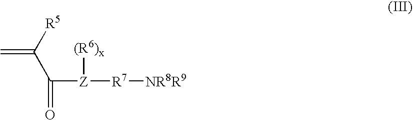
where R⁵, R⁶, independently, are a hydrogen atom or a methyl radical, R⁷ is an alkylene radical having 1 to 24 carbon atoms, optionally substituted by alkyl radicals, and R⁸, R⁹ are C₁-C₂₄ alkyl radicals. Z is a nitrogen atom together with x=1 or is an oxygen atom together with x=0,
(b) 22 to 97.98% by weight of at least one water-soluble monomer chosen from N-vinyllactams,
(c) 0 to 50% by weight of at least one further free-radically copolymerizable monomer,
(d) 0.01 to 10% by weight of at least one crosslinking monomer having at least two ethylenically unsaturated, nonconjugated double bonds, and
(e) 0.01 to 10% by weight of at least one regulator
(ii) subsequent quaternization or protonation of the polymer if the monomer (a) used is a nonquaternized monomer or an only partially quaternized monomer.

15. A process for the preparation of the polymers by free-radically initiated copolymerization of a monomer mixture of
(a) 2 to 70% by weight of at least one cationic monomer or quaternizable monomer chosen from the group consisting of diallylamines of the formula (II) in which R⁴ is C₁-C₂₄-alkyl 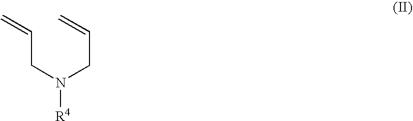
and N,N-dialkylaminoalkyl acrylates and methacrylates and N,N-dialkylaminoalkylacrylamides and -methacrylamides of the formula (III), 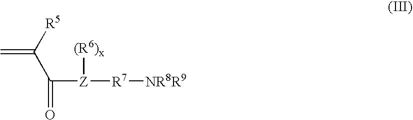
where R⁵, R⁶, independently, are a hydrogen atom or a methyl radical, R⁷ is an alkylene radical having 1 to 24 carbon atoms, optionally substituted by alkyl radicals, and R⁸, R⁹ are C₁-C₂₄-alkyl radicals. Z is a nitrogen atom together with x=1 or is an oxygen atom together with x=0,
(b) 22 to 97.98% by weight of at least one water-soluble monomer chosen from N-vinyllactams,
(c) optionally at least one further free-radically copolymeriazable monomer,
(d) at least one crosslinking monomer having at least two ethylenically unsaturated, nonconjugated double bonds, in the presence of a regulator (e) and subsequent quaternization or protonation of the polymer, if the monomer (a) is a nonquaternized monomer or an only partially quaternized monomer.

16. (canceled)

17. (canceled)

18. Cosmetic preparation comprising a polymer according to claim 7.

19. Cosmetic preparation containing as conditioning agent a polymer according to claim 7.

20. The cosmetic preparation of claim 2 where derivatives of the formula (I), in which R¹ to R³ are hydrogen, C₁-C₄-alkyl or phenyl, are used as monomer (a)

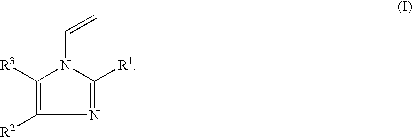

21. The cosmetic preparation of claim 2 where N-vinyllactams are used as monomer (b).

22. The cosmetic preparation of claim 2 where thiols are used as regulator.
